(19) United States
(12) Patent Application Publication
(10) Pub. No.: US 2016/0213277 A1
(43) Pub. Date: Jul. 28, 2016

(54) Programmable Multimodal Stimulator for Cortical Mapping

(71) Applicant: PHOENIX CHILDREN'S HOSPITAL, INC., Phoenix, AZ (US)

(72) Inventor: Rémy Wahnoun, Phoenix, AZ (US)

(21) Appl. No.: 14/971,621

(22) Filed: Dec. 16, 2015

Related U.S. Application Data
(60) Provisional application No. 62/096,337, filed on Dec. 23, 2014.

(51) Int. Cl. A61B 5/0484; A61B 5/04; A61B 5/00

(52) U.S. Cl. CPC A61B 5/0484; A61B 5/04842; A61B 5/04845; A61B 5/0051; A61B 5/0057; A61B 5/04012; A61B 5/4064

(57) ABSTRACT

A system allowing for sensory stimulation, includes a programmable handheld device for visual, auditory and mechanical stimulation of the body, and associated computer software to analyze cortical evoked responses and map relevant areas on a brain sketch or rendering. The device generates single or series of pulses of programmable intensity/frequency and duration. It can be used by itself to manually stimulate modalities of interest, connected to a cortical signal amplifier as an external event source in order to record simultaneously the stimulation events and the cortical data for later analysis, plugged to a recording computer to show in real time activated cortical areas, or used for cognitive studies.

### BACKGROUND AND FIELD OF THE INVENTION

1. Field of the Invention

The present invention relates to functional mapping of the brain for diagnostic and pre-surgical planning purposes. In surgical planning for epileptic focus resection, for example, functional mapping of eloquent cortex is attained through direct electrical stimulation of the brain. This procedure is uncomfortable, can trigger seizures or nausea, and relies on subjective evaluation.

### 2. Background

Approximately 65 million individuals worldwide are living with epilepsy, 2.2 million of whom are in the United States (Epilepsy Foundation 2013). The first and most common form of relief relies on anti-epileptic drugs. However, one fourth to one-third of the cases do not become seizure-free from drug therapy alone (Privitera; Téllez-Zenteno et al. 2005). In these situations, surgery may be an option if a single, localizable focus can be identified and safely removed. Generalized seizures, arising within and rapidly engaging bilaterally distributed networks, or seizures localized in the language areas of the brain may not be resectable and therefore surgical strategies would more likely entail disconnection to interrupt seizure spread through the network or alternatively, neuromodulation. To obtain a broad sense of the origins and types of seizures, neural activity is first monitored using an electroencephalography (EEG) system through scalp recordings of brain activity (Phase I). If the seizures appear to be potentially focal and unilateral, surgically implanted electrocorticographic (ECoG) electrodes on the cortical surface, or depth electrodes for deep foci, are used to monitor cortical activity during seizures and further define the epileptic foci (Phase II). The decision for surgical resection or intervention depends on the data from this invasive monitoring strategy clearly delineating the epileptogenic zone and ensuring that resection of the seizure foci will not significantly impact neurological functions. During Phase II monitoring, in addition to seizure localization, several procedures are then used to define areas of eloquent cortex and attempt to estimate the functions possibly affected by the surgical procedure.

### Standard Sensorimotor Mapping

Electrical Cortical Stimulation (ECS) is considered the gold standard for sensorimotor functional delineation of eloquent tissue in the brain. In contrast to continuous monitoring where the electrical current from the brain is recorded, during ECS, electrical current is passed between neighboring electrodes to evoke sensory or motor manifestations. Typically, during ECS, 50 Hz square pulse trains are applied lasting two to five seconds (Ikeda et al. 2002). The stimulation current is gradually increased up to 10 mA, until a sensory response, a motor response, or an after-discharge is elicited. A bottom-up approach can also be performed by electrically stimulating peripheral nerves and visually observing evoked responses in the cortical signals. These methods result in the construction of a somatotopic map of sensory and motor function.

However those two techniques have limitations. The somatic response is subjective and interpretative based on the patient's response and/or direct visualization by the tester. For sensory areas, it is often difficult to interpret evoked stimuli. In children, particularly those that are too young or non-verbal due to cognitive dysfunction, interpretation of sensation can be very difficult. Additionally, after-discharges are frequently an unwanted consequence of ECS stimulation, and can lead to seizures. Unfortunately, stimulation evoked seizures have little diagnostic value: they do not show a strong correlation with natural seizure foci (Blume et al. 2004).

Cortical stimulation does not always elicit motor responses on children under 10 years of age (Connolly et al. 2010). In addition, sensory mapping relies often on the patient's ability to describe sensations or follow directions, which is often dramatically lowered as the patients are recovering from the ECoG implantation during the invasive monitoring period. Cortical mapping can be uncomfortable and trigger nausea or seizures: at best this can prolong the process considerably, at worst it can result in a termination of clinical mapping for the day.

### Somatosensory Pathway

Vibratory information is relayed by the lemniscal pathway from the cutaneous mechanoreceptors to the somatosensory cortical areas (Patestas and Gartner 2006; Cruccu et al. 2008). The pathway of somatosensory information is still debated, with evidence leaning towards a serial organization from the thalamus to the primary (SI, posterior wall of the central sulcus) then secondary (SII, upper bank of the Sylvian fissure) somatosensory cortices (Inui et al. 2004; Kalberlah et al. 2013): aside from this clear serial stream, both nociceptive and non-nociceptive information are largely processed in parallel streams (Garraghty 2007; Liang and Iannetti 2011). After activation of mechanoreceptors, a strong response can be observed in the contralateral SI, followed by a bilateral response in SII (Simões et al. 2001). Finger representation in the contralateral SI covers a 10-20 mm long strip (Penfield and Boldrey 1937; Pollok et al. 2002; Overduin and Servos 2004), following a latero-medial distribution, from the thumb to the small finger with a limited amount of overlap (Simões et al. 2001; Schweizer et al. 2008; Severens 2008), and notable inter-individual variability (Schweizer et al. 2008). The volume of cortical representation of the digits shows some relative correlation to the receptor density of the fingers, and is larger for the thumb than the index, and ring fingers (Overduin and Servos 2004). SII does not seem to follow a topological organization of the fingers (Kalberlah et al. 2013) or show a strong spatial overlap (Ruben et al. 2001; Simões et al. 2001), and may be involved in bimanual tasks (Simões et al. 2001).

### Time Domain Analysis

Time domain analysis typically centers on evoked potentials, averaging cortical responses over a large amount of trials. This procedure enhances time locked components, and reduces the impact of non-related activity. Somatosensory Evoked Potentials (SEPs) can be elicited by stimulating peripheral nerve fibers (Allison 1987) and are conventionally named after their polarity followed by their latency in milliseconds. In an MEG based study (Simões et al. 2001), Simões et al located three current dipoles elicited by air stimulation of the fingers; A P66 in contralateral SI, followed by a P100 in contralateral SII, and P111 in ipsilateral SII. At the sensor level, evoked fields showed a postcentral contralateral P52 and N96, reversing polarity in precentral locations. In the post-Rolandic contralateral EEG, notable somatosensory responses to short tactile pulses consist of the sequence P50, N70, P100, N140, then P300 (Hämäläet al. 1990; Eimer and Forster 2003). N140 is largest in the contralateral cortex and often presents a bifid peak shape. Vibratory stimulation exhibits similar responses, but with a larger P100 as compared to tactile pulses (Hämäläinen et al. 1990).

Time-Frequency Domain Analysis

Time-frequency domain analysis expands the analysis of evoked responses in terms of changes in oscillatory activity induced by stimulation. Evoked Response Synchronization (ERS) and Evoked Response Desynchronization (ERD) correspond to an increase or decrease of the power of oscillations in a given band. The general assumption is that ERS emerges as the result of a surge of concurrent activity in a network, while ERD arises as the result of a decreased correlation (Neuper and Pfurtscheller 2001). It has also been hypothesized that smaller functional networks may exhibit higher ERS frequencies than larger areas (Singer 1993; Pfurtscheller et al. 2001). Somatosensory stimulation has been shown to result in an increase of oscillatory activity in the Gamma range, as well as a decrease in Alpha and Beta bands in the contralateral postcentral cortex (Pfurtscheller et al. 2001; Fukuda et al. 2008, 2010), with hints that the initial processing of the stimuli may initiate in the high frequency domain (Fukuda et al. 2010). Some of these induced oscillations are phase-locked to the stimulus and appear concurrently with SEP components. Both phase-locked and non-phase-locked oscillations are believed to be present in sensorimotor studies (Fukuda et al. 2010).

During movement preparation, a contralateral ERD (below 30 Hz) is followed by bilateral ERD and associated contralateral ERS (above 30 Hz) during execution. Finally, around 700 ms after movement onset, a beta resynchronization signals a return to baseline (Salmelin et al. 1995; Pfurtscheller and Lopes da Silva 1999; Pfurtscheller et al. 2001, 2003).

### SUMMARY OF THE INVENTION

The present disclosure provides an alternative approach, involving vibratory stimulation of individual fingers and a cluster based analysis in the time-frequency domain, to avoid the above deficiencies. The invention generates tactile, visual, and auditory stimulation for cortical mapping as well as sensory assessment. The programmable multimodal stimulator can be used in standalone mode, plugged to a recording device (EEG, EMG . . . ), or to a computer and used in conjunction with its associated software. It is placed next to the ears, facing the eyes, or anywhere on the surface of the skin. It delivers pulse sequences of light, sounds, and/or mechanical stimuli (vibration or taps) to the subject.

In standalone mode, it can be used with a questionnaire to assess sensory health (i.e. after traumatic brain injury, stroke or spinal cord injury). In conjunction with its associated software, it can be connected to a computer and cortical signals amplifier (e.g. EEG, ECoG) to record evoked responses, and to generate in real time a map of the cortical areas directly on the subject's brain, or on a brain template. Its output can be connected as an input of another device to synchronize the stimulation with other signals (i.e. for conduction studies). It can be programmed for cognitive studies, for example in tasks where subjects are asked to respond to specific light, sound or vibration patterns by pushing specific buttons.

A device according to one aspect of the invention offers three types of stimulation, a manual and an automatic mode, and an output for simultaneous recording with other information. The associated software allows automatic mapping of cortical responses into a brain sketch or rendering. Applications of the invention include:

Standalone Mode: Users can manually stimulate the subjects by activating the Visual, Auditory, or Tactile switches. This can be used for example to quickly assess function loss after traumatic brain injury, stroke, or spinal cord injury.
As a Stimulus Source: The output connector can be used to record the stimulation alongside other data streams. For example, recording the stimulus synchronously with EMG activity can be used for conduction studies. It can be plugged directly to an EEG amplifier as an event source, and allow post hoc analysis of evoked responses.
Real Time Mapping: With the associated software, and plugged to an EEG amplifier, the device can be used to build a real time somatosensory map of the brain.
Cognition: The device could also be programmed for cognitive studies. For example, a task can be created for which the subject will press on of the three buttons according to specific LED colors, sounds, or vibration patterns.

### BRIEF DESCRIPTION OF THE DRAWINGS

FIG. 1 is a perspective diagram of a multimodal stimulator in accordance with one embodiment of the invention;

FIG. 2 is a schematic block diagram of functional components of a multimodal stimulator in accordance with one embodiment of the invention;

FIG. 3 shows example output display information for a user of the present invention;

FIG. 4 shows pulse sequence response parameters for triggering different modalities of the stimulator according to the present invention.

### DETAILED DESCRIPTION OF THE INVENTION

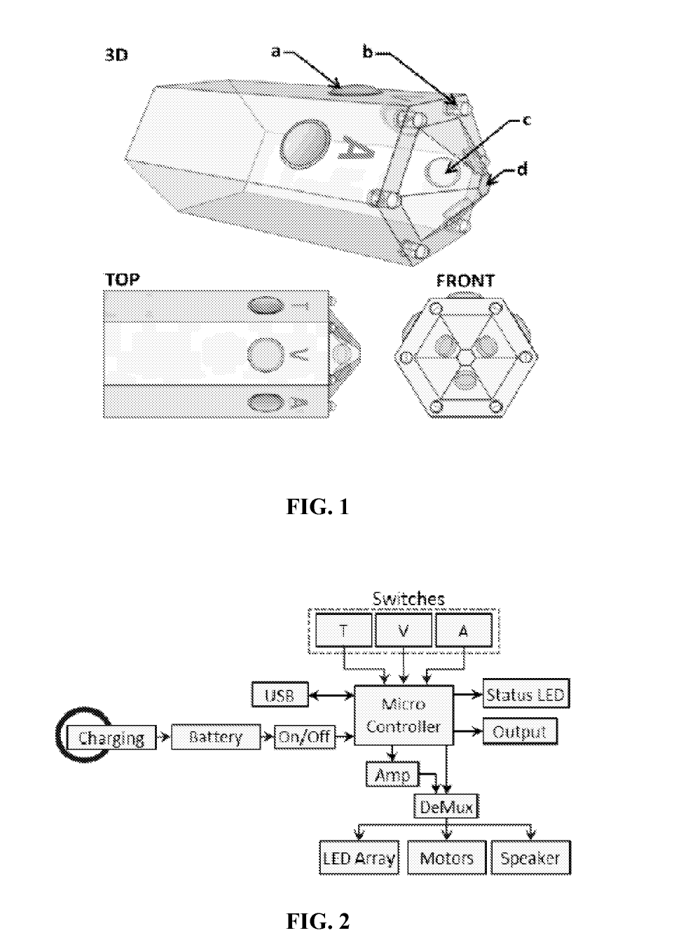

Referring to the example embodiment of FIG. 1, the overall shape of the device is a hexagonal prism, prolonged by a hexagonal pyramid. The device is battery powered and can be inductively charged. Three momentary switches (a), positioned on three contiguous faces of the prism, activate manual stimulation, and are labeled V, T, and A, to trigger respectively Visual, Tactile, and Auditory stimulation. Visual Stimulation is generated by six RGB LEDs placed around the base of the pyramid (b). The LEDs can be programmed to display any combination of red, green and blue. Tactile Stimulation is transferred from the device to the subject through the tip of the hexagonal pyramid (d). Vibrations can be generated from one or more offset weight motors (c), or from linear resonant actuators. Tapping can be obtained by replacing the motors with solenoid actuators. In this configuration, a prong extruding from the tip strikes the subject's skin. Auditory stimulation is generated from a loudspeaker embedded in the device.

Referring to FIG. 2, the microcontroller used is an Atmega 328, chosen for its versatility and programmability. A multiplexer (DeMux) allows selecting the effector (LEDs, Motors, or Speaker). The amplifier circuit (Amp) ensures sufficient current is provided to the effectors (transistor), and includes a capacitor and a rectifier diode to absorb voltage spikes.

The back panel includes an On/Off switch, a status LED (Green for full battery, Orange for half full, Red for critical battery), a female micro USB plug control from a computer, and programming. A Dual (ground+signal) keyhole touchproof connector is provided to trigger events on other devices (i.e. recording computer, EEG amplifier).

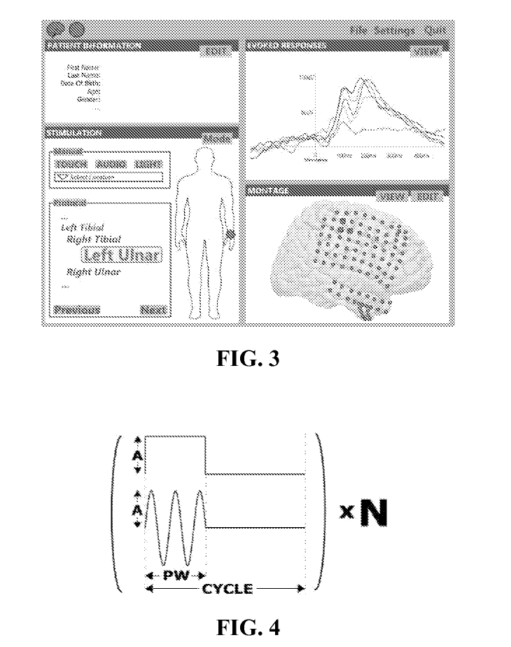

Referring to FIG. 3, the device-associated software can be used on a computer for manual or guided stimulation, or to build in real-time a map of sensory areas on a brain model or sketch. Standard analysis will be provided (e.g. P300 detection); Users will be able to develop plugins for their specific goals. The example software as shown includes 5 panels:

Patient Information, which can be edited after clicking on the EDIT button.
Stimulation, where the user can switch between the automatic and manual mode by clicking on the MODE button, displaying either the Manual or Protocol subpanels. In the manual mode, the user can choose stimulation modality and location. In the Protocol mode, protocols are defined as sequences of stimulations which can include one or more modalities. The user can load, save and edit protocols. The emphasized text and mark on the body sketch show the current stimulation. The PREVIOUS and NEXT buttons can be used to navigate the protocol.
Evoked responses displays the cortical evoked responses after stimulation. The VIEW button allows switching between time and time-frequency displays, and select which electrodes are displayed.
Montage shows the electrode configuration on the patient brain. VIEW allows switching to different views (3d, flat, grid). EDIT allows to load/edit/save configurations. As different stimuli are elicited, this view will highlight the electrodes with significant responses, building a somatosensory map of the patients' brain. Template montages such as 10/10 10/5 and 10/20 configurations will be provided.

The top row shows the status icons (left) indicating whether the software is connected to the stimulator and the cortical amplifier, and different options (right). The FILE button allows loading, editing, and saving sessions and protocols.

### Programming

The different modalities are triggered using the same principle.

Referring to FIG. 3, a Pulse is defined by an ON period consisting of a basic waveform (sinusoidal, triangular, sawtooth, or square) followed by an OFF period. Pulse parameters include waveform type, amplitude (FIG. 4, A), pulse width (FIG. 4, PW), cycle duration (FIG. 4, CYCLE), and frequency (if needed by the waveform).

A Pulse Sequence comprises one or more (N) individual pulses.

A Protocol is a file containing series of lines describing pulses sequences, modality, and location. Numeric parameters can be given specific values or ranges (to allow random stimulations), as:

| Line | Location | Modality | Number of Pulses | Waveform | A (%) | PW (ms) | CYCLE (ms) | Frequency (Hz) |
|---|---|---|---|---|---|---|---|---|
| 1 | Left Hand | Tactile | 10 | SQU | 100 | 100-300 | 50-1500 | |
| 2 | Right Hand | Tactile | 10 | SQU | 100 | 100-300 | 50-1500 | |
| 3 | Left Ear | Auditory | 10 | SIN | 100 | 250 | 50-1500 | 440-880 |
| 4 | Right Ear | Auditory | 10 | SIN | 100 | 250 | 50-1500 | 440-880 |

### Applications

Application 1: Standalone Mode

The device can be used by itself by pressing any of the three momentary switches to generate single pulses of Visual, Tactile, or Auditory stimuli. For example, the device can be programmed to deliver by default a white light pulse, a steady vibration/tap, or a 440 Hz sound, depending on the activated switch.

The device can be placed on the subject's skin at different locations for tactile testing, or facing an eye or ear. The subject can then be asked to respond if he/she senses the stimulation. One application of this mode could be to rapidly test sensory function after spinal cord injury, traumatic brain injury, or stroke.

### Application 2: As A Stimulus Source

The output connector can be connected to an external device (i.e. EEG amplifier) as an event source in order to synchronize the stimulus condition with other signals (i.e. cortical data), allowing post hoc analysis (i.e. ERP studies, brain computer interfaces).

The device's output can also be connected to an amplifier for conduction/EMG studies. In this configuration, the device triggers a vibration or tapping stimulus and the conduction speed between the stimulation and recording locations can be estimated.

This device can also be used to generate signals for different experiments/assessments. For example, a subject can be asked to hold the device and respond to specific colors or vibrations patterns by pressing one of its three buttons.

### Application 3: Real Time Mapping

For this application, a computer is connected to both the device (through USB connection) and to an EEG amplifier. The user can follow a specific protocol (see Software section) to generate in real time a map of the activated cortical areas under the different stimuli.

A standard session for somatosensory mapping follows the sequence:
1. Connect the device and amplifier to the recording computer, and then wait for the status icons to turn green.
2. Fill in patient information
3. Load/Edit the montage
4. Load a protocol
5. For each protocol condition, place the stimulator in the intended location (skin area, eye, or ear) and press any of the three buttons. The computer will generate stimuli (random pulses, trains . . . ) until significant response, or a maximum stimulation time is reached.
6. The user can navigate through the protocol using the PREVIOUS and NEXT buttons to reiterate or skip conditions or observe recorded data.
7. Results are saved in an automatically generated report showing all stimulations and evoked responses. Evoked responses are individually recorded for post/hoc analysis.

1. A multimodal sensory stimulator system, comprising:
a handheld device configured to provide stimulation of visual, auditory and tactile senses of a patient, and
at least one processor configured to analyze cortical evoked responses of said patient to stimulation provided by said handheld device, and map relevant areas on a graphical representation of the brain of said patient associated with said evoked responses.

2. The system of claim 1, wherein said device generates a single stimulatory pulse of programmable intensity/frequency and duration

3. The system of claim 1, wherein said device generates a series of pulses of programmable intensity/frequency and duration.

4. The system of claim 1, wherein said device is a standalone device used configured to manually stimulate modalities of interest.

5. The system of claim 1, wherein said device is connected to a cortical signal amplifier as an external event source in order to simultaneously record stimulation events and cortical data for later analysis.

6. The system of claim 1, wherein said device is connected to a recording computer to show in real time activated cortical areas, or used for cognitive studies.

7. The system of claim 1, wherein said handheld device comprises:
a hexagonal prism;
a plurality of momentary switches positioned on contiguous faces of said prism, each of said switches manually activating sensory stimulation to respectively trigger visual, tactile, and auditory stimulation of said patient.

8. The system of claim 1, wherein said handheld device comprises a plurality of LEDS to provide visual stimulation of said patient.

9. The system of claim 1, wherein said handheld device comprises at least one offset weight motor to provide vibratory tactile stimulation of the patient.

10. The system of claim 1, wherein said handheld device comprises at least one solenoid actuator to provide tapping tactile stimulation of the patient.

11. The system of claim 1, wherein said handheld device comprises at least loudspeaker to provide auditory stimulation of the patient.

12. The system of claim 7 wherein said handheld device comprises a plurality of LEDS mounted around a base of said prism to provide visual stimulation of said patient.

13. The system of claim 7, wherein said handheld device comprises at least one offset weight motor mounted on a pyramidal tip of said prism to provide vibratory tactile stimulation of the patient.

14. The system of claim 7, wherein said handheld device comprises at least one solenoid actuator mounted on a pyramidal tip of said prism to provide tapping tactile stimulation of the patient.

15. The system of claim 7, wherein said handheld device comprises at least loudspeaker mounted in a surface of said prism to provide auditory stimulation of the patient.

16. The system of claim 1, wherein said handheld device comprises at least one linear resonant actuator to provide vibratory tactile stimulation of the patient.

17. The system of claim 7, wherein said handheld device comprises at least one linear resonant actuator mounted on a pyramidal tip of said prism to provide vibratory tactile stimulation of the patient.

18. The system of claim 1, wherein said handheld device is programmable to provide sensory stimulation via visual, tactical or auditory stimulators triggered by pulse trigger signals.
